(19) United States
(12) Patent Application Publication
(10) Pub. No.: US 2020/0129124 A1
(43) Pub. Date: Apr. 30, 2020

(54) GLASSES COMPLIANCE MONITOR AND ASSOCIATED METHOD

(71) Applicant: ARIZONA BOARD OF REGENTS ON BEHALF OF THE UNIVERSITY OF ARIZONA, Tucson, AZ (US)

(72) Inventors: Erin M. HARVEY, Tucson, AZ (US); Joseph M. MILLER, Tucson, AZ (US); Martha J. MOHLER, Tucson, AZ (US); Janet M. ROVEDA, Tucson, AZ (US)

(73) Assignee: Arizona Board of Regents on Behalf of the University of Arizona, Tucson, AZ (US)

(21) Appl. No.: 16/319,747

(22) PCT Filed: Jul. 21, 2017
(86) PCT No.: PCT/US2017/043359

§ 371 Date: Jan. 22, 2019

Related U.S. Application Data
(60) Provisional application No. 62/365,869, filed on Jul. 22, 2016.

(51) Int. Cl. A61B 5/00; A61H 5/00; G02C 11/00; G02C 11/06

(52) U.S. Cl. CPC A61B 5/6814; A61H 5/00; G02C 11/10; A61H 2201/5082; A61B 2562/0223; A61H 2201/5084; G02C 11/06

(57) ABSTRACT

A compliance monitor for wear on a head includes a data logger with processor, and memory containing firmware; with sensors that may include one or more of magnetometers, accelerometers, gyroscopes, and temperature sensors. The firmware is adapted to process sensor readings to determine intervals where the compliance monitor is attached to a person's head, and in embodiments is adapted to scan for signatures indicating donning of the compliance monitor. The method of determining wear of a head-mounted device by a person includes determining differences between a magnetic field measured at the start of a time interval and the time interval, differences between a temperature reading at a sensor on a head side of the head-mounted device and on an ambient side of the head-mounted device, accelerations of the head-mounted device indicative of wear, and rotations of the head-mounted device indicative of wear.

### RELATED APPLICATIONS

This application claims priority to U.S. Patent Application Ser. No. 62/365,869, filed Jul. 22, 2016, and Patent Cooperation Treaty Application Number PCT/US17/43359, filed Jul. 21, 2017. Both of these applications are incorporated herein by reference.

### BACKGROUND

Eyeglasses may be prescribed for multiple reasons, some of which, like correction of amblyopia or relief of headaches, require consistent wear by patients to be effective. While many patients comply because they see better while wearing eyeglasses, young patients treated for amblyopia may feel they see worse when fitted with eyeglasses with an integral eyepatch, and thus may resist treatment. Young patients may also resist wearing either eyeglasses or orthodontic headgear because of a perceived effect of these devices on appearance.

Patient-reported medical treatment histories are not always accurate. Patients may intentionally or unintentionally misstate hours of wear of eyeglasses or other devices such as helmets for those having had recent skull surgery, removable orthodontic appliances such as headgear, and hearing aids. Inaccurate data makes it difficult for a physician to determine the degree to which a patient complies with a treatment plan.

Advertisers may wish to keep track of head position of members of sample audiences watching their ads, as may researchers who seek insights into other aspects of vision and psychology. In particular, advertisers may wish to document percentages of time a person is both wearing a tracking device and the person's head is oriented towards a screen.

### SUMMARY OF THE EMBODIMENTS

In an embodiment, a compliance monitor is configured for, or disposed within a device configured for, wear on a head of a person. The compliance monitor includes a data logger and a sensor subsystem. The data logger includes a processor and a memory containing firmware. The firmware includes machine-readable instructions adapted to be executed by the processor. The sensor subsystem includes at least one sensor selected from the group consisting of magnetometers, accelerometers, gyroscopes, and temperature sensor, the sensor subsystem communicatively coupled to the data logger. The data logger firmware in embodiments is adapted to process readings of the sensor to determine intervals where the compliance monitor is attached to a person's head, and in embodiments is adapted to scan for signatures indicating donning of the compliance monitor

In an embodiment, a method of determining wear of a head-mounted device by a person includes determining
(a) a difference between an ambient magnetic field measured at the start of a time interval and measured at the end of the time interval,
(b) a difference between a temperature reading at a temperature sensor on a head side of the head-mounted device and an ambient side of the head-mounted device,
(c) accelerations of the head-mounted device indicative of wear, and
(d) rotations of the head-mounted device indicative of wear.

In one embodiment, a glasses compliance monitor (GCM) is disclosed. The GCM includes a data logger, a magnetometer communicatively coupled to the data logger, and mounting hardware configured to mechanically couple the data logger and the magnetometer to an eyeglass frame.

In another embodiment, another glasses compliance monitor is disclosed. The second glasses compliance monitor includes an eyeglass frame, a data logger, and a magnetometer communicatively coupled to the data logger. The data logger and magnetometer are integrated into the eyeglass frame.

In another embodiment, a method for determining use of eyeglasses by an individual is disclosed. The method includes a step determining a first motion indicator as a difference between an ambient magnetic field, proximate the eyeglasses, measured at the start of a first time interval and measured at the end of the first time interval. The method also includes a step of determining a second motion indicator as a difference between the ambient magnetic field as measured at the start and as measured at the end of a second time interval that (a) is temporally separated from the first time interval by a gap period, and (b) does not overlap the first time interval. The method also includes a step of determining whether the person either removed or put on the eyeglasses, according to the gap period and magnitudes of the first and second motion indicators.

### BRIEF DESCRIPTION OF THE FIGURES

FIG. 1 shows an exemplary use scenario of a compliance monitor attached to an eyeglass frame, in an embodiment.

FIG. 2 is a schematic diagram of a compliance monitor, which is an example of the compliance monitor of FIG. 1.

FIG. 3 shows a magnetic field record, which is an example of magnetic field record stored in memory of the compliance monitor of FIG. 2.

FIG. 4 is a graph illustrating motion-amplitude change data of the magnetic field record of FIG. 3.

FIG. 5 is a calibration dataset processed from the magnetic field record of FIG. 3.

FIG. 6 is a flowchart illustrating an exemplary method for determining use of eyeglasses by an individual, in an embodiment.

FIG. 7 is a block diagram illustrating another embodiment of a compliance monitor adapted for use with devices worn upon a person's head.

FIG. 8 is a flowchart illustrating an embodiment of a method for determining wear of the compliance monitor by a person using multiple sensors of the compliance monitor.

FIG. 9 is a flowchart illustrating an alternative embodiment of a method for determining wear of the compliance monitor by a person.

### DETAILED DESCRIPTION OF THE EMBODIMENTS

A small, wearable compliance monitor and data logger device may be comfortably affixed either to a subject's head directly, as with a headband or hat, or to a head-worn device, such as spectacles. When worn with spectacles, the compliance monitor may be either directly attached to the frame of the spectacles or, for example, to a band on the back of the head attached to each earpiece of the spectacle temples. In some embodiments, the small, wearable data logger is used to track head position over time. This information is useful for such purposes as recording human head motion patterns of a device attached to spectacles, which indicates periods of time that glasses were being worn as well as what the wearer is paying attention to. For vision research, this provides a measure of compliance to facilitate studies of the beneficial effects of spectacle wear on vision and child development. For treatment monitoring, an electronic compliance record provides a measure of compliance allowing a physician to consider alternative treatment or apply persuasion when compliance is insufficient to meet treatment goals.

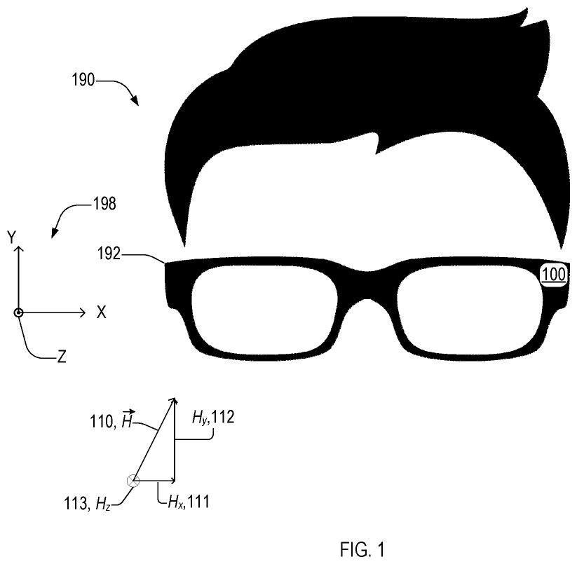

FIG. 1 shows an exemplary use scenario of a compliance monitor 100 attached to eyeglass frames 192 worn by a person 190. Compliance monitor 100 is configured to record head postures of person 190. Person 190 is on Earth, which has a geomagnetic field Ft with components Hₓ, H_{y}, and H_{z} with respect to a coordinate system 198. Herein, geomagnetic field \vec{ } H and its components Hₓ, H_{y}, and H_{z} are also referred to as geomagnetic field 110 and field components 111, 112, and 113, respectively. Herein, directions x, y, and z refer to directions x, y, and z of coordinate system 198 unless otherwise indicated.

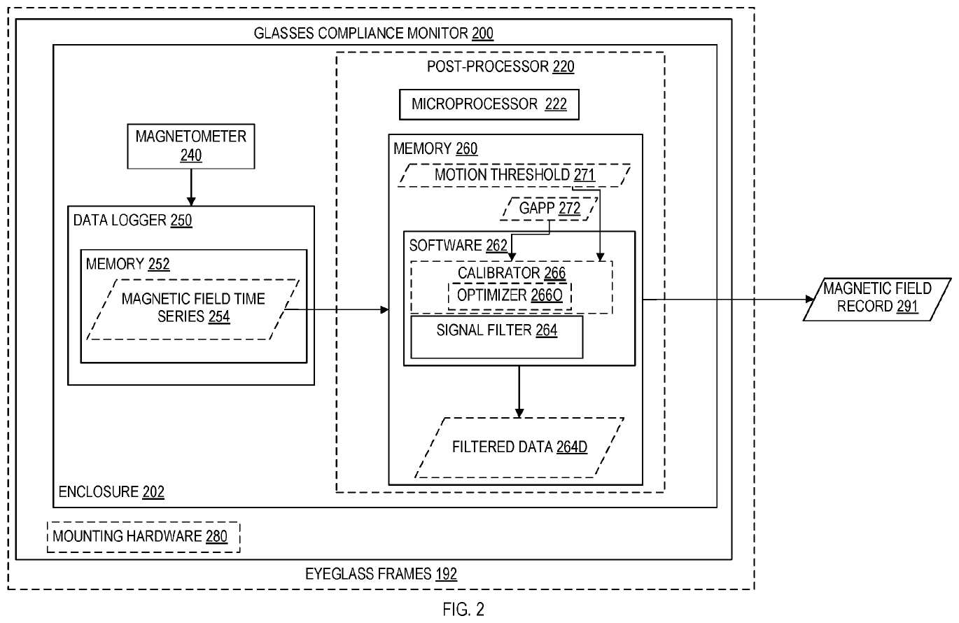

FIG. 2 is a schematic diagram of a compliance monitor 200, which is an example of compliance monitor 100 of FIG. 1. Compliance monitor 200 includes a magnetometer 240, a data logger 250, and optionally a post-processor 220, and an enclosure 202. Compliance monitor 200 may be incorporated into eyeglass frames 192. Data logger 250 includes memory 252, which stores a magnetic-field time series 254 received from magnetometer 240. Post-processor 220 may be separate from compliance monitor 200, for example, as part of a computer configured to receive magnetic-field time series 254 from compliance monitor 200, and may in some embodiments link to the compliance monitor via wireless communication (e.g., Bluetooth, IEEE 802.11 (Wi-Fi), wireless body area network), or wired communication (e.g., USB).

Post-processor 220 includes a memory 260 and a microprocessor 222. Memory 260 includes firmware or software 262, which includes signal filter 264. Compliance monitor 200 may also include mounting hardware 280 for affixing to an eyeglass frame or other head-mounted device, such as eyeglass frames 192. Mounting hardware 280 is, for example, configured to affix compliance monitor 200 to the temple or earpiece of eyeglass frames 192. Mounting hardware 280 may, in an embodiment, be configured to attach compliance monitor 200 to a cord or strap connected to eyeglass frames 192, wherein the cord connects to each ear piece of frames 192 and enables frames 192 to hang from a neck of person 190, or secures frames 192 such that they do not accidently fall off.

Memory 252 and 260 may each represent one or both of volatile memory such as SRAM, DRAM, or any combination thereof, and nonvolatile memory such as FLASH, ROM, magnetic media, optical media, or any combination thereof and may include removable memory such as mini-SD cards. Memory 260 may be part of memory 252.

Magnetic-field time series 254 may include a time series of each field component 111, 112, and 113 of geomagnetic field 110. Signal filter 264 generates filtered data 265 from magnetic-field time series 254. Filtered data 265 may be proportional to a time-derivative of magnetic-field time series 254. Signal filter 264 may implement a band-pass filter, such as a finite impulse response (FIR) high-pass filter, on magnetic-field time series 254 to generate filtered data 265, to pass a predetermined passband of temporal frequencies of magnetic-field time series 254. For example, a passband spanning 0.33 and 1.0 Hz may be useful for isolating changes in magnetometer measurements associated with a target action, such as removing or putting on eyeglasses.

Other target actions, and associated applications of compliance monitor 200, may include: recording head postures assumed by subjects with normal eye movements doing specific tasks, to assist in more ergonomic design of both workstations and eyeglasses appropriate to the workstation. Applications of compliance monitor 200 may include assisting in the design of progressive bifocal lenses, and recording head postures assumed by subjects with limitations of normal eye motion, such as nystagmus, in order to document type and frequency of anomalous head postures. For vision research, the compliance monitor may assist in design of the most appropriate surgical or spectacle intervention for a patient with abnormal eye movements.

Compliance monitor 200 outputs magnetic field record 291, which includes at least one of magnetic-field time series 254 and filtered data 265. In an embodiment in which compliance monitor 200 does not include post-processor 220, magnetic field record 291 includes magnetic-field time series 254, and a computing device separate from compliance monitor 200 (such as one including post-processor 220) computes filtered data 265.

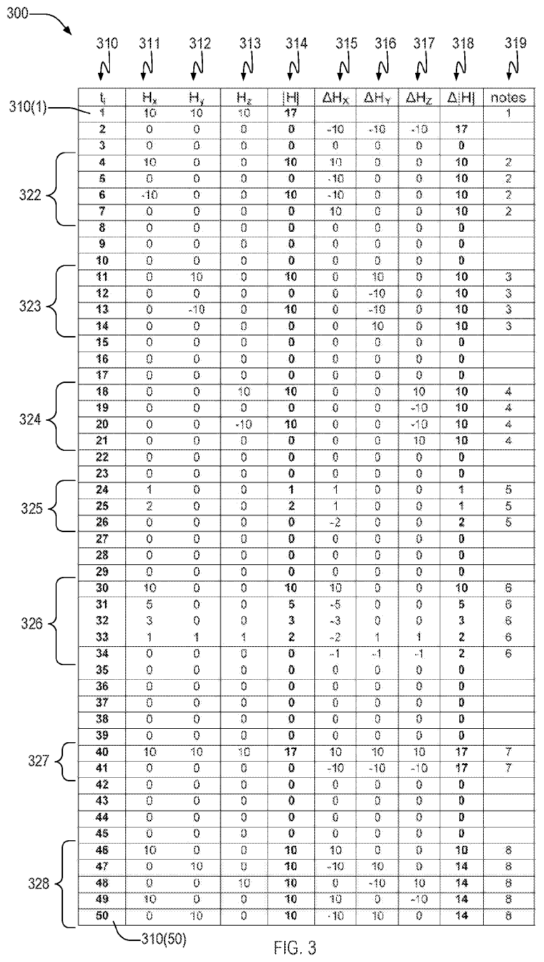

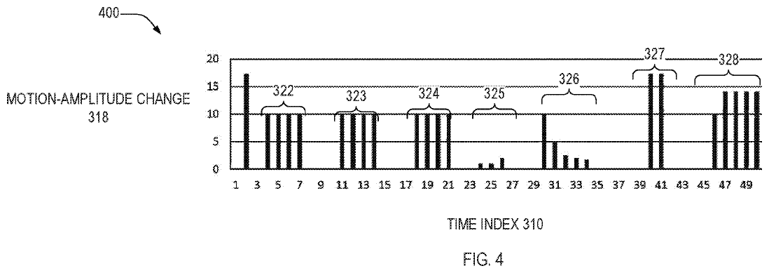

FIG. 3 shows a magnetic field record 300, which is an example of magnetic field record 291. Magnetic field record 300 includes time indices 310(1-50), field components 311, 312, and 313, motion amplitudes 314, field-change components 315, 316, and 317, and field-magnitude changes 318. FIG. 4 is a graph 400 illustrating field-magnitude changes 318 as a function of time indices 310.

Field components 311, 312, and 313 are, for example, proportional to magnetic field components 111, 112, and 113, respectively, as measured by magnetometer 240, and are an example of magnetic-field time series 254. Each time index 310 in a row denotes a respective time t₁, t₂, . . . , t₅₀ at which measurements of field components 311, 312 and 313, in the row, were recorded. Each of motion amplitudes 314 is a quadrature sum of the corresponding field components 311, 312, and 313. Each of field-change components 315, 316, and 317 at a time index 310(m) is a difference between a respective field component 311, 312, and 313 at time index 310(m) and the same field component at time index 310(m−1). For example, field-change component 315(4)=311(4)−311(3)=10. Field-magnitude changes 318 are similarly related to motion amplitudes 314.

Magnetic field record 300 includes time-intervals 322, 323, 324, and 327 corresponding to time-intervals t₄-t₇, t₁₁-t₁₄, t₁₈-t₂₁, and t₄₀-t₄₁, respectively. Time-intervals 322, 323, 324, and 327 are separated by gap periods Δt₁=t₁₁−t₇, Δt₂=t₁₈−t₁₄, Δt₃=t₄₀−t₂₁. Each of time-intervals 322, 323, 324, and 327 corresponds to a different type of head motion by person 190. Time-interval 322 corresponds to chin turn from left to right (±x direction). Time-interval 323 corresponds to chin nodding (±y direction) by person 190. Time-interval 324 corresponds to head tilt by person 190. Time-interval 327 corresponds to head motion of person 190 when person 190 sneezed.

Herein, non-zero values of field-change components 315, 316, and 317, and field-magnitude changes 318 are referred to as motion indicators. For example, within time-intervals 322, 323, 324, or 327 and at a time index 310, at least one of (i) field-change components 315, 316, and 317, and (ii) field-magnitude changes 318 is non-zero, and hence is an example of a motion indicator. Of field-magnitude changes 318, examples of motion indicators are non-zero values include field-magnitude changes 318(4-7), 318(11-14), 318(18-21), and 318(40-41).

Magnetic field record 300 optionally includes a notes column 319, which is included here for illustrative purposes. Note “1” at time index 310(1) indicates impulse noise at startup. Notes 5, 6, and 8 of respective time-intervals 325, 326, and 328 correspond to motion of person 190.

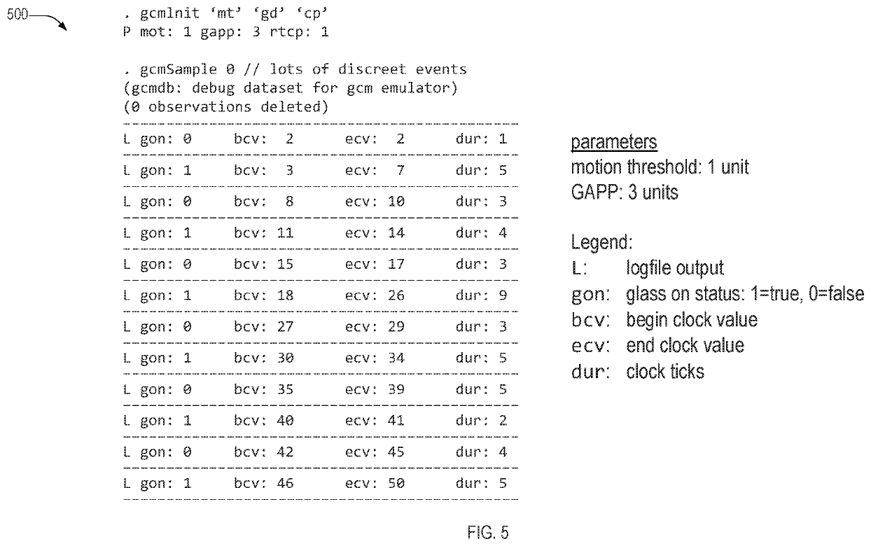

FIG. 5 is a calibration dataset 500 processed from magnetic field record 300 by an embodiment of compliance monitor 200. Calibration dataset is, for example, produced by calibrator 266 stored in memory 260 of GCM 200. Calibration dataset 500 includes predictions of when the bespectacled state of person 190 changed during the time-interval between times t₁ and t₅₀. The first bespectacled state of calibration dataset 500 is that person 190 was not wearing any glasses, e.g., frames 192, at time t₁, as indicated by glasses-on-status (or “bespectacled status”) gon=0 at bcv=2 (bcv denotes begin clock value). Calibration dataset 500 is based on two parameters: a motion threshold parameter 271 (“mot”) and an inter-motion interval 272 (“gapp”), each of which may be stored in memory 260. Calibration dataset 500 is an output of an execution of calibrator 266 with motion threshold parameter 271 and inter-motion interval 272 set to one unit (mot=1) and three units (gapp=3), respectively. In calibration dataset 500, a “unit” corresponds to 0.2 seconds.

In the example of calibration dataset 500, motion threshold parameter 271 corresponds to field-magnitude changes 318 of magnetic field record 300. Motion threshold parameter 271 may involve quantities in addition to, or instead of, field-magnitude changes 318, such as one or more field-change components 315, 316, and 317. For example, the motion of eyeglass frames 192 when being removed or put on may correspond to a distinctive change in the geomagnetic field component 112 and corresponding measured field components 312.

With mot=1 and gapp=3, calibration dataset 500 predicts a change of glasses-on-status gon at times indicated by bcv. The accuracy of these predictions may be compared with direct observation of person 190 during the observation period corresponding to time indices 310. Calibrator 266 may include an optimizer 267 for finding the values of motion threshold parameter 271 and inter-motion interval 272 that include a calibration dataset 500 with minimized errors in predicted changes in glasses-on-status. Optimizer 267 may employ a computational optimization technique known in the art.

In an embodiment, calibrator 266 includes a third motion threshold indicative of minimum and/or maximum time duration of a field-component change 315-317 or a field-magnitude changes 318. For example, calibration dataset 500 predicts changes of bespectacled status at time indices 310(40-42), which may correspond to when person 190 merely sneezed, rather than also having removed or put on frames 192. A motion-duration threshold of, for example, three units would disqualify a value of field-magnitude changes 318 in time-interval 327 from being a candidate for a change in bespectacled status.

Optimal values of motion threshold parameter 271 and inter-motion interval 272 involve tradeoffs between (a) minimizing false positive detections (bespectacled status change) when motion threshold parameter 271 and interval 272 are too low, and (b) minimizing missed detections of bespectacled status change when threshold parameter 271 and inter-motion interval 272 are too high. In embodiments, at least one of motion threshold parameter 271 and inter-motion interval 272 are specifically optimized to detect a negative change in bespectacled status, that is, when frames 192 are removed from person 190. Alternatively, motion threshold parameter 271 and inter-motion interval 272 may be specifically optimized to detect a positive change in bespectacled status, that is, when frames 192 are placed on person 190.

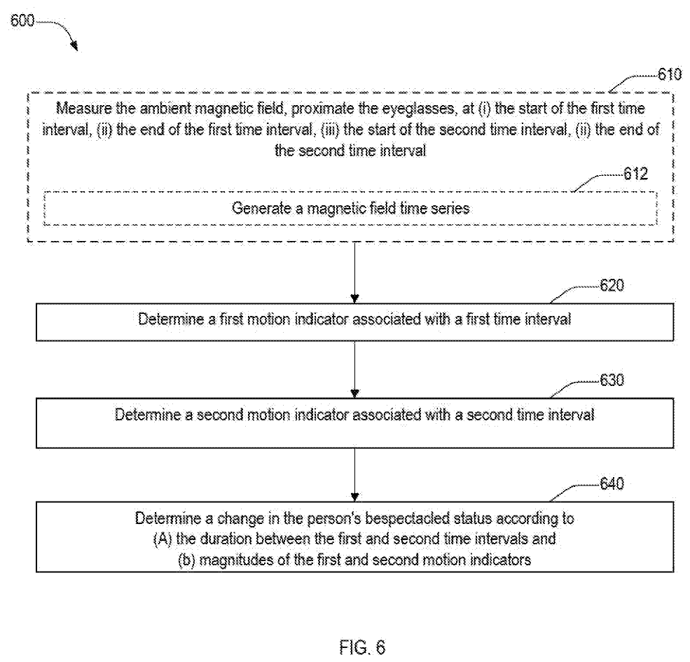

FIG. 6 is a flowchart illustrating an exemplary method 600 for determining use of eyeglasses by an individual. Method 600 is, for example, implemented by software 262 of post-processor 220, FIG. 2.

Method 600 includes steps 620,630, and 640. Method 600 may also include a step 610, which may include a step 612.

Step 610 includes measuring the ambient magnetic field at (i) the start of a first-time-interval, (ii) the end of the first-time-interval, (iii) the start of a second time-interval, (and (iv) the end of the second time-interval. In an example of step 610, magnetometer 240 measures geomagnetic field 110 proximate to eyeglass frames 192 at one or more time-intervals 322, 323, and 324. Step 610 may include step 612, in which method 600 generates a magnetic-field time series. In an example of step 612, compliance monitor 200 generates a magnetic-field time series 254.

Step 620 includes determining a first motion indicator as a difference between the ambient magnetic field measured at the start of the first time-interval and measured at the end of the first time-interval. In an example of step 620, post-processor 220 determines a first motion indicator to be field-magnitude change 318(7) corresponding to time-interval (t₇−t₆).

Step 630 includes determining a second motion indicator as a difference between the ambient magnetic field as measured at the start and measured at the end of a second time interval that (a) is temporally separated from the first time-interval by a gap period, and (b) does not overlap the first time-interval. In an example of step 630, post-processor 220 determines a second motion indicator to be field-magnitude change 318(11) corresponding to a time-interval (t₁₁−t₁₀).

Step 640 includes determining a change in the person's bespectacled status according to the gap period and magnitudes of the first and second motion indicators. The change in bespectacled status may be either a positive change (glasses-off to glasses-on) or a negative change (glasses-on to glasses-off). In an example of step 640, post-processor 220 determines a change in the bespectacled status of person 190, with respect to eyeglass frames 192, according to field-magnitude changes 318(7) and 318(11) and gap period Δt₁=t₁₁−t₇ therebetween. Gap period Δt₁=4, which corresponds to the “clock ticks” value (dur) in the fourth row of calibration dataset 500, FIG. 5, where glasses-on status (gon) changes from zero to one because the gap period exceeds a predetermined inter-motion interval 272 (gapp=3) and both field-magnitude changes 318(7,11) exceed the motion threshold parameter 271 (mot=1).

In embodiments, additional sensors, such as accelerometers or gravity sensors, may complement magnetometer 240. The magnetic sensor and accelerometer complement each other because the magnetic field is perpendicular to the gravitational field to which the accelerometer responds in part, and so they complement each other. For people who are either standing or sitting with their spine more or less perpendicular to the floor, lateral head motion will induce more change in the magnetic field than the gravitational field. For chin motion parallel to the spine (e.g., up and down motions), the gravitational field will be more sensitive to change. There would be advantages to including both measurements in the system.

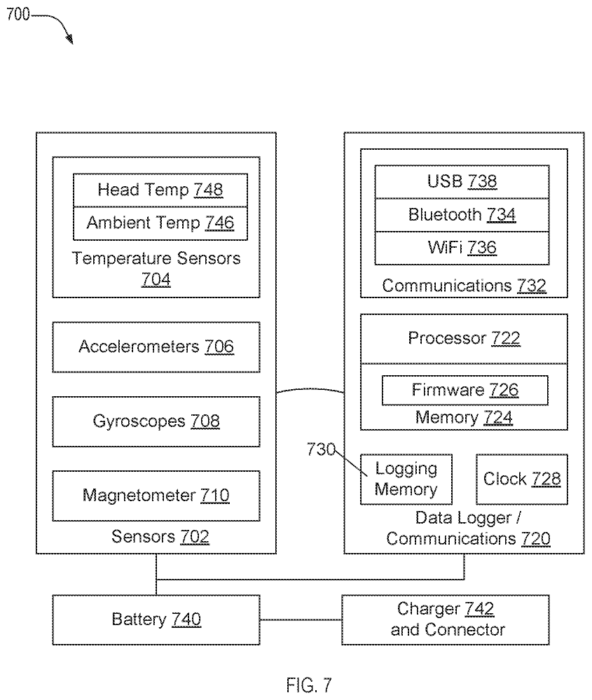

In alternative embodiments, as illustrated in the block diagram of FIG. 7, a compliance monitor 700 has sensors 702 that may include one or more additional sensors such as temperature sensors 704, accelerometers 706, or gyroscopes 708 that may supplement or replace magnetometer 240. In the embodiment of FIG. 7, sensors 702 couple to, and may be in the same physical assembly as data logger/communications unit 720. Data/logger/communications unit 720 in an embodiment includes a small microprocessor 722 and memory 724. Memory 724 includes firmware 726. Microprocessor 722 is coupled to or contains a clock circuit 728 adapted to provide a date and time-of-day to microprocessor 722. Microprocessor 722 is also coupled to a data-logging memory 730, which in some embodiments is a nonvolatile memory and may be a removable memory such as a mini-SD card. In some embodiments, microprocessor 722 is also coupled to a communications unit 732.

Memory 724 and 730 may be transitory and/or non-transitory and may represent one or both of volatile memory such as SRAM, DRAM, computational RAM, other volatile memory, or any combination thereof, and non-volatile memory such as FLASH, ROM other non-volatile memory, or any combination thereof. Memory 730 may be transitory and/or non-transitory and may represent one or both of volatile memory such as SRAM, DRAM, computational RAM, other volatile memory, or any combination thereof, and writeable non-volatile memory such as FLASH, EEPROM, other non-volatile memory, or any combination thereof. Part or all of memory 724 and 730 may be integrated into microprocessor 722.

In embodiments, communications unit 732 includes one or more digital radios such as a Bluetooth digital radio 734, an IEEE 802.11-compatible (Wi-Fi) digital radio 736, and other digital radios such as those compatible with body-area digital networks. Communications unit 732 may also include a wired communications port, such as a Universal Serial Bus (USB) port 738, which may also double as a battery charger connection.

Compliance monitor 700 also includes a battery 740 coupled to provide power to the sensors 702 and data logger 720. In some embodiments, battery 740 is rechargeable and a charger 742 and charging connector is also provided to recharge battery 740.

In an embodiment, temperature sensors 704 include a pair of temperature sensors: ambient temperature sensor 746 and head temperature sensor 748. Head temperature sensor 748 is configured to be mounted on a head surface of compliance monitor 700, the head surface being a portion of the compliance monitor adapted for wear adjacent to skin of a person wearing compliance monitor 700. Ambient temperature sensor 746 is mounted on an exterior surface of compliance monitor 700, where it is exposed to ambient air. Since a person's typical body core temperature is between 98° F. and 100° F., and ambient air temperatures are often much cooler, a temperature difference between sensors 746 and 748 may indicate that compliance monitor 700 is being worn by a person. This temperature difference is referred to herein as a differential temperature. Differential temperatures may in some embodiments be particularly useful for distinguishing between a person carrying compliance monitor 700 in a backpack or suitcase and the person wearing the compliance monitor and associated device on his or her head. In an alternative embodiment, where the exterior and head surfaces are identical, compliance monitor 700 can determine a head surface as the surface where the sensed temperature is closest to physiological temperature.

In an alternative embodiment, ambient sensor 746 is omitted with the head-side temperature sensor 748 present in the compliance monitor 700. In this embodiment: (i) temperatures in the physiological range indicate that a person is wearing compliance monitor 700, (ii) a shift in head-side temperature towards physiological skin temperatures indicates that compliance monitor 700 is being put on by the person, (iii) and a shift in head-side temperature away from physiological skin temperature indicates that compliance monitor is no longer 700 is being worn by the person.

Table 1 provides an experimental indication that temperature differences between sensor 746 and sensor 748 provide reasonably effective indication that, using a particular set of thresholds, all four sensor types, magnetometer 710, accelerometer 706, gyroscope 708, and temperature sensors 704, can individually distinguish some instances of a person wearing that sensor type on their head from the same sensor type when it is not being worn. The listed percentages indicate the percentages of samples above respective thresholds for mild activity in a particular dataset. The results of Table 1 were obtained using 180 seconds of data recorded in each condition and analyzed using fixed thresholds that have not been fully optimized to distinguish mild activity while being worn from no activity. Dataset A corresponds to high activity without temperature sensor in contact with skin. Dataset B corresponds to no activity, without temperature sensor in contact with skin. Dataset C corresponds to low activity with temperature sensor in contact with skin. Dataset D corresponds to high activity with temperature sensor in contact with skin.

TABLE 1
Percent of samples above respective thresholds for mild activity

| | Magnetometer | Accelerometer | Difference in Temp |
|---|---|---|---|
| A: high activity, | 49% | 54% | 0% |
| no skin contact | | | |
| B: no activity, no | 1% | 0% | 0% |
| skin contact | | | |
| C: low activity, | 3% | 4% | 100% |
| skin contact | | | |
| D: high activity, | 38% | 53% | 100% |
| skin contact | | | |

In another dataset, as indicated in Table 2, a prototype embodiment was shown to have 68% sensitivity, detecting more than two-thirds of times when compliance monitor 700 was being worn, and 81% specificity.

TABLE 2
Compliance Monitor Sensitivity

| | compliance monitor worn | compliance monitor not worn |
|---|---|---|
| Log: on | 894 | 249 |
| Log: off | 423 | 1,047 |
| Σ | 1,317 | 1,296 |
| Sensitivity | 68% | — |
| Specificity | — | 81% |

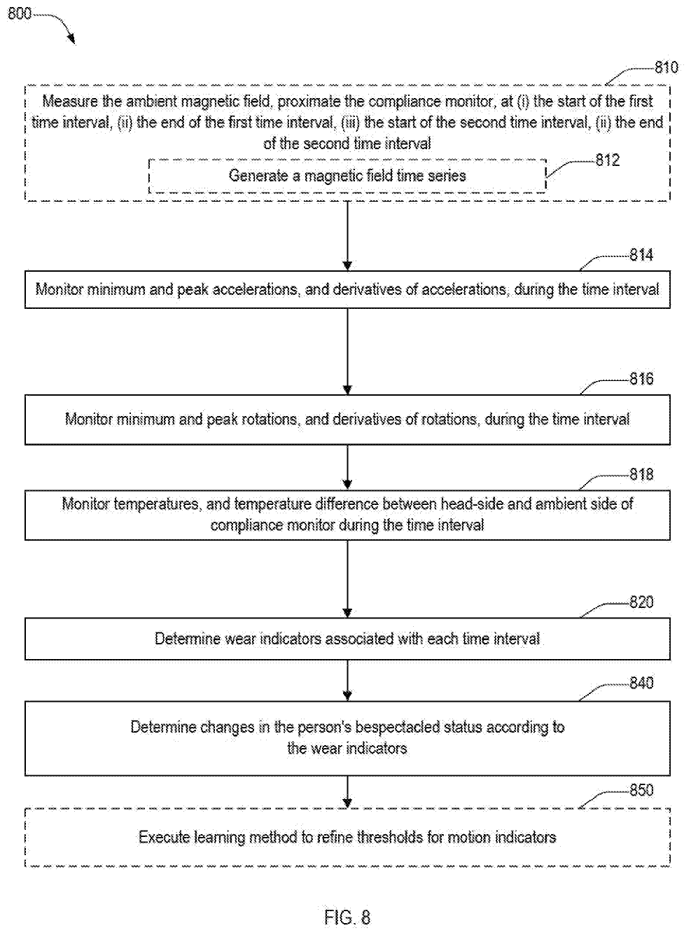

The embodiment of FIG. 7 may in a particular embodiment be operated according to a method 800 of FIG. 8. FIG. 8 is a flowchart illustrating method 800 for determining wear of eyeglasses by an individual. Method 800 may be implemented by firmware 726 of microprocessor 722, FIG. 7, or by a postprocessor, of any embodiment having multiple sensor types such as the combination of magnetometer 710, temperature sensors 704, accelerometers 706, and gyroscopes 708 of sensors 702 of FIG. 7.

Method 800 begins with measuring 810 the ambient magnetic field at the start and end of each time-interval, including the first and second time-intervals. Step 810 may include a step 812, in which method 800 generates a magnetic-field time series. In an example of step 812, compliance monitor 700 generates a magnetic-field time series.

In addition to magnetic field, method 800 includes measuring 814 minimum, maximum, and peak rates of change, or first derivatives of summed accelerations and gravitational fields at the compliance monitor during each time-interval. Similarly, method 800 includes measuring 816 minimum, maximum, and peak rates of change (derivatives) of rotations at the compliance monitor during each time interval; and method 800 includes measuring 818 temperature on the head-side of the compliance monitor and measuring a temperature difference between the head-side of the compliance monitor and an ambient temperature at the compliance monitor during each time-interval.

Once sensors are read, wear indicators are determined 820 from sensed values including changes in the magnetic-field time series from the first to the second time-interval, the minimum, maximum, and peak rates of change of summed accelerations and gravitational fields, minimum, maximum, and peak rates of change of rotations, and temperature and temperature difference between the head-side of the compliance monitor and an ambient temperature at the compliance monitor. In determining the wear indicators, in an embodiment microprocessor 722 compares each of these sensed values to prior sensed values and to a baseline using dynamic thresholds and, in some embodiments, an artificial intelligence function such as a neural network. In an alternative embodiment, microprocessor 722 determines if each of these measured values or their rates of change exceed a threshold, generating a flag if the value or rate of change exceeds the threshold. Then, the flags are logically OR-ed together to generate an overall wear indicator.

Step 840 includes determining a change in the wear status of the compliance monitor and a current wear status, including in eyeglass embodiments a bespectacled status, according to wear indicators. Step 850 includes executing a learning method to refine thresholds for the wear indicators.

Step 850 is one of executing a learning method to refine thresholds for the wear indicators.

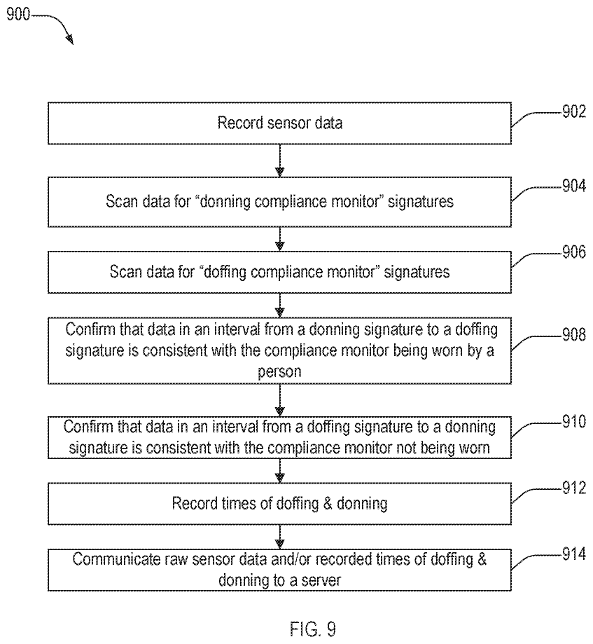

In an alternative embodiment, firmware 726 directs compliance monitor 700 to operate according to method 900 of FIG. 9. In this method, the compliance monitor records 902 sensor data from all sensors with which it may be equipped, including, in a particular embodiment, the combination of magnetometer 710, temperature sensors 704, accelerometers 706, and gyroscopes 708 of sensors 702 of FIG. 7.

Once data is recorded in a recorded data list, the monitor scans 904 the data-, seeking “donning compliance monitor” signatures associated with attaching the compliance monitor to a person's head, that in an embodiment include a period that may be motionless, and in an embodiment accelerations, rotations, and temperature changes that are expected to occur when a person picks up the monitor and attaches it to his or her head, followed by a period where at least an occasional motion or differential temperature is found in the recorded data list.

In a particular embodiment, the signature for donning the device consists of a ten-second period of inactivity in the data logger as represented by no suprathreshold activity in channels associated with the magnetometer, accelerometer, and temperature sensor. This period of inactivity is followed by a period of activity as indicated by the magnetometer and accelerometer, followed by an increase of temperature if only a head-side temperature sensor is provided and ambient temperature was less than physiologic temperature, or development of a differential temperature between head-side and ambient temperature sensors. Such temperature changes may lag behind sensed activity in accelerometer, gyroscope, and magnetometer signals. The period of inactivity may deviate from ten seconds without departing from the scope hereof. For example, the period of inactivity may be between eight seconds and twenty seconds.

The monitor also scans 906 the data set for “doffing compliance monitor” signatures, that may that in an embodiment include a period that may include occasional small motions and temperature differences, followed by in an embodiment accelerations, rotations, and temperature changes that are expected to occur when a person removes the eyeglasses 192 from his or her head, followed by a period where an absence of occasional motion or differential temperature is found in the recorded data.

In a particular embodiment, the doffing compliance monitor signature is determined by sensing a period of activity in accelerometer, gyroscope, and/or magnetometer signals, followed by a ten-second period of inactivity in these channels accompanied by a decrease of temperature if only a head-side temperature sensor is provided and ambient temperature less than physiologic temperature, or reduction of a differential temperature between head-side and ambient temperature sensors. The period of activity may deviate from ten seconds without departing from the scope hereof. For example, the period of activity may be between eight seconds and twenty seconds.

The compliance monitor firmware may then confirm 908 that data in an interval from a donning signature to a doffing signature is consistent with the compliance monitor being worn by a person, and confirm 910 that data in an interval from a doffing signature to a donning signature is consistent with the compliance monitor not being worn.

Once data is logged, times of confirmed donning and doffing are recorded 912 in logging memory 730.

In embodiments, recorded data, either prior to evaluation or after periods of patient wear of the compliance monitor are determined by evaluating recorded sensor data, is stored in logging memory 730 until it is communicated 914 to a server or user application on a computing device by communications unit 732.

In embodiments, the compliance monitor is attached to or embedded within an eyeglasses frame. In an alternative embodiment, the compliance monitor is attached to or embedded within a helmet, such as a bicycle helmet or a post-surgery skull-protection helmet. In another alternative embodiment, the compliance monitor is attached to or embedded within a hearing-aid. In another alternative embodiment, the compliance monitor is attached to or embedded within a headgear attached to an orthodontic appliance.

In additional alternative embodiments, the compliance monitor is incorporated within one of virtual reality goggles, and a glasses-mounted or helmet-mounted heads-up display. In such embodiments, the magnetometer, accelerometer, and gyroscopes serve to track changes in orientation by a person wearing the goggles/display, such that the display can be updated to compensate for the person's movements. Such an embodiment may be useful in combat aircraft.

In another alternative embodiment, the compliance monitor 200 is additionally equipped with a tracking device adapted to determine a location of a wearing person. Applications for such an embodiment include data collection for one of television ratings, advertising, and psychological studies. In such an embodiment, the person's orientation as determined by the magnetometer and accelerometers may be combined with the person's location to determine times when the person is both wearing the compliance monitor and has head pointed towards, and thus is likely to be paying attention to, a television or video screen.

In yet another embodiment, the compliance monitor is used in medical studies of workplace human factors and effectiveness of workplace accommodations to determine the extent and character of head and neck movements in those who have complained of neck pain while working with computer monitors.

### Combinations of Features

The features herein described may be present in various combinations, some of which may be summarized here.

In an embodiment designated A, A compliance monitor is configured for, or disposed within a device configured for, wear on a head of a person. The compliance monitor includes a data logger and a sensor subsystem. The data logger includes a processor and a memory containing firmware. The firmware includes machine-readable instructions adapted to be executed by the processor. The sensor subsystem includes at least one sensor selected from the group consisting of magnetometers, accelerometers, gyroscopes, and temperature sensor, the sensor subsystem communicatively coupled to the data logger.

In a particular embodiment designated AA of the compliance monitor designated A, the compliance monitor is attached to or disposed within an eyeglass frame.

In a particular embodiment designated AB of the compliance monitor designated A or AA, the sensor subsystem includes a magnetometer.

A particular embodiment designated AC of the compliance monitor designated A, AA, or AB the sensor subsystem includes a temperature sensor on a head surface of the compliance monitor, adapted for wear adjacent to skin of the person's head, and a temperature sensor on an ambient side of the compliance monitor.

In a particular embodiment designated AD of the compliance monitor designated A, AA, AB, or AC the sensor subsystem comprises an accelerometer.

In a particular embodiment designated AE of the compliance monitor designated A, AA, AB, AC, or AD the firmware is adapted to process readings of the sensor to determine intervals where the compliance monitor is attached to a person's head.

In a particular embodiment designated AF of the compliance monitor designated A, AA, AB, AC, AD, or AE, the firmware is adapted to scan for signatures indicating donning of the compliance monitor.

In a particular embodiment designated AG of the compliance monitor designated A, AA, AB, AC, AD, AE, or AF, the firmware is configured to direct the processor to execute the machine-readable instructions to execute a method of an embodiment designated by one of B, BB, BC, and BD.

In an embodiment designated B, a method of determining wear of a head-mounted device by a person includes determining at least one wear indicator as a wear indicator selected from the group consisting of a difference between an ambient magnetic field measured at the start of a time interval and measured at the end of the time interval, a difference between a temperature reading at a temperature sensor on a head side of the head-mounted device and an ambient side of the head-mounted device, accelerations of the head-mounted device indicative of wear, and rotations of the head-mounted device indicative of wear. The method also includes determining periods of wear of the head-mounted device from the wear indicators.

In a particular embodiment designated BA of the embodiment designated B, the wear indicator comprises a plurality of wear indicators selected from the group consisting of a difference between an ambient magnetic field measured at the start of a time interval and measured at the end of the time interval, a difference between a temperature reading at a temperature sensor on a head side of the head-mounted device and an ambient side of the head-mounted device, accelerations of the head-mounted device indicative of wear, and rotations of the head-mounted device indicative of wear.

In a particular embodiment designated BB of the embodiment designated B or BA, the method includes verifying periods of wear and non-wear for consistency with sensor readings during those intervals.

In a particular embodiment designated BC of the embodiment designated B, BA, or BB, the method includes communicating the periods of wear of the head-mounted device to a server.

In a particular embodiment designated BD of the embodiment designated B, BA, BB, or BC; the wear indicator includes a difference between a temperature reading at a temperature sensor on a head side of the head-mounted device and an ambient side of the head-mounted device.

In an embodiment designated C1, a glasses compliance monitor includes a data logger, a magnetometer communicatively coupled to the data logger, and mounting hardware configured to mechanically couple the data logger and the magnetometer to an eyeglass frame. The embodiment designated C1 may also include a temperature sensor communicatively coupled to the data logger, the mounting hardware being further configured to mechanically couple the temperature sensor to the eyeglass frame.

In an embodiment designated C2, glasses compliance monitor includes an eyeglass frame, a data logger integrated into the eyeglass frame, and a magnetometer integrated into the eyeglass frame and communicatively coupled to the data logger. The embodiment designated C2 may also include a temperature sensor integrated into the eyeglass frame and communicatively coupled to the data logger.

In an embodiment designated D a method for determining use of eyeglasses by a person, includes: determining a first motion indicator as a difference between an ambient magnetic field, proximate the eyeglasses, measured at the start of a first time interval and measured at the end of the first time interval; determining a second motion indicator as a difference between the ambient magnetic field measured at the start and measured at the end of a second time interval that (a) is temporally separated from the first time interval by a gap period, and (b) does not overlap the first time interval; and determining a change in the person's bespectacled status according to the gap period and magnitudes of the first and second motion indicators.

In a particular embodiment designated DA of the embodiment designated D, the step of determining a change in the person's bespectacled status includes determining the change in the person's bespectacled status when (a) the first and the second motion indicators each exceed a predetermined magnitude, and (b) the gap period exceeds a predetermined duration.

In a particular embodiment designated DB of the embodiment designated D or DA, the method includes measuring the ambient magnetic field at (i) the start of the first time interval, (ii) the end of the first time interval, (iii) the start of the second time interval, (ii) and the end of the second time interval.

In a particular embodiment designated DC of the embodiment designated D, DA, or DB, the step of measuring includes measuring the ambient magnetic field at a plurality of times, including the start and end of the first time interval and the second time interval, to generate a magnetic field time-series; and the steps of determining the first and second motion indicator include time-differentiating the magnetic field time-series.

In a particular embodiment designated DD of the embodiment designated D, DA, DB, or DC the step of time-differentiating includes applying a finite impulse-response high-pass filter to the magnetic field time series.

In a particular embodiment designated DE of the embodiment designated D, DA, DB, DC, or DD, the method includes determining the gap period exceeding both the first time interval and the second time interval.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A compliance monitor configured for, or disposed within a device configured for wear on a head of a person comprising:
a sensor subsystem including at least one of a magnetometer, an accelerometer, a gyroscope, and a temperature sensor;
a data logger communicatively coupled to the sensor subsystem and including a processor and a memory storing machine-readable instructions that, when executed by the processor, control the processor to:
repeatedly determine a wear indicator that includes at least one of (a) a difference between an ambient magnetic field measured with the magnetometer at the start of a time-interval and measured at the end of the time-interval, (b) a difference between temperature readings measured with the temperature sensor on a head-side of the device and an ambient-side of the device, (c) accelerations of the device, measured with the accelerometer, indicative of wear, and (d) rotations of the device, measured with the gyroscope, indicative of wear; and
determine periods of wear of the device from the determined wear indicator.

2. A compliance monitor comprising the device of claim 1, wherein the compliance monitor is attached to or disposed within an eyeglass frame.

3. (canceled)

4. The compliance monitor of claim 1, the sensor subsystem including a first temperature sensor on a surface of the compliance monitor adapted for wear adjacent to skin of a person's head and a second temperature sensor on an ambient-side of the compliance monitor.

5. The compliance monitor of claim 1, the device including an eyeglass frame, the sensor subsystem including one of a magnetometer and a temperature sensor.

6. The compliance monitor of claim 1, further comprising machine-readable instructions stored in the memory that, when executed by the processor, control the compliance monitor to detect intervals where the compliance monitor is attached to a person's head.

7. The compliance monitor of claim 1, further comprising machine-readable instructions stored in the memory that, when executed by the processor, control the compliance monitor to detect signatures indicating donning of the compliance monitor.

8. (canceled)

9. A method for determining wear of a head-mounted device by a person, comprising:
repeatedly determining a wear indicator selected from the group consisting of (a) a difference between an ambient magnetic field measured at the start of a time-interval and measured at the end of the time-interval, (b) a difference between a temperature reading at a temperature sensor on a head-side of the head-mounted device and an ambient-side of the head-mounted device, (c) accelerations of the head-mounted device indicative of wear, and (d) rotations of the head-mounted device indicative of wear; and
determining periods of wear of the head-mounted device from the determined wear indicators.

10. (canceled)

11. The method of claim 9, further comprising verifying periods of wear and non-wear for consistency with sensor readings during those intervals.

12. The method of claim 9 further comprising communicating the periods of wear of the head-mounted device to a server.

13. The method of claim 8, wherein the wear indicator comprises the difference between the temperature reading at the temperature sensor on the head-side of the head-mounted device and an ambient-side of the head-mounted device.

14.-17. (canceled)

18. A method for determining use of eyeglasses by a person, comprising:
determining a first motion indicator as a difference between an ambient magnetic field, proximate to the eyeglasses, measured at a start of a first time-interval and measured at an end of the first time-interval;
determining a second motion indicator as a difference between the ambient magnetic field measured at a start and measured at an end of a second time-interval that (a) is temporally separated from the first time-interval by a gap period, and (b) does not overlap the first time-interval; and
determining a change in the person's bespectacled status according to the gap period and the first and second motion indicators.

19. The method of claim 18, determining a change in the person's bespectacled status comprising:
determining when (a) each of the first motion indicator and the second motion indicator exceed a predetermined magnitude, and (b) the gap period exceeds a predetermined duration.

20. (canceled)

21. The method of claim 18, further comprising
generating a magnetic-field time series by (i) measuring the ambient magnetic field at a first plurality of times, including the start and end of the first time-interval and (ii) measuring the ambient magnetic field at a second plurality of times, including the start and end of the second time-interval, the magnetic-field time series including the first and second plurality of times and respective corresponding measured ambient magnetic fields;
the determining the first and second motion indicators further comprising time-differentiating the magnetic-field time series.

22. The method of claim 21, the time-differentiating comprising applying a finite-impulse-response high-pass filter to the magnetic-field time series.

23. The method of claim 18, further comprising determining the gap period exceeding both the first time-interval and the second time-interval.

24. The compliance monitor of claim 1, further comprising the device, at least one of the sensor subsystem and the data logger being attached to or embedded within the device.

25. The compliance monitor of claim 24, the device including a hearing aid.

26. The compliance monitor of claim 24, the device including a helmet.

27. The compliance monitor of claim 24, the device including a headgear attached to an orthodontic device.
